(19) United States
(12) Patent Application Publication
(10) Pub. No.: US 2023/0285047 A1
(43) Pub. Date: Sep. 14, 2023

(54) INTRODUCTION DEVICE FOR THE PERCUTANEOUS INTRODUCTION OF DRAINAGE TUBES, IN PARTICULAR THORACIC DRAINAGE TUBES

(71) Applicant: REDAX S.P.A., POGGIO RUSCO (IT)

(72) Inventor: Lucio GIBERTONI, MIRANDOLA (IT)

(21) Appl. No.: 18/018,432

(22) PCT Filed: Jul. 19, 2021
(86) PCT No.: PCT/EP2021/070160

§ 371 Date: Jan. 27, 2023

(30) Foreign Application Priority Data
Jul. 28, 2020 (IT) 102020000018193

(51) Int. Cl. A61B 17/34; A61B 17/3209

(52) U.S. Cl. CPC A61B 17/3415; A61B 17/3496; A61B 17/3209

(57) ABSTRACT

An introduction device for the percutaneous introduction of drainage tubes, in particular thoracic drainage tubes, including:
a main body, which has a grip portion adapted to be gripped by a hand of an operator, and a proximal portion provided with an internal longitudinal cavity;
a terminal tubular element, which is extended longitudinally and distally from the proximal portion of the main body;
a longitudinally extended percussion element, which can slide within the terminal tubular element between a retracted position and an extracted position; and
a tubular introducer which forms inside it a through duct and can be configured in a coupled cutting configuration, in which the tubular introducer is fitted on the terminal tubular element, and in an uncoupled configuration, in which the tubular introducer and the terminal tubular element are uncoupled.

### CROSS-REFERENCE TO RELATED APPLICATIONS

The present application is a 35 U.S.C. § 371 National Stage patent application of International patent application PCT/EP2021/070160, filed on 19 Jul. 2021, which claims priority to Italian patent application 102020000018193, filed on 28 Jul. 2020.

### TECHNICAL FIELD

The present disclosure relates to an introduction device for the percutaneous introduction of drainage tubes, in particular thoracic drainage tubes.

The percutaneous introducer according to the present disclosure is mainly but not exclusively useful and practical in the medical field, in particular for the introduction of surgical drainage tubes which are flexible and have various profiles into organic tissues.

The percutaneous introducer according to the present disclosure therefore finds particular application in the medical practice of thoracic drainage, the goal of which is the evacuation of fluids, i.e., air and/or liquids, from the thoracic cavity of a patient in order to avoid their accumulation. Typically, said fluids form as a consequence of traumas and/or surgical procedures.

### BACKGROUND

In general, a surgical thoracic drainage tube is a flexible tube provided by using transparent and sterile plastic materials, such as for example PVC (polyvinyl chloride) or silicone; said tube is inserted, through an incision, in the thorax of the patient until it reaches the pleural space in the thoracic cavity, in order to act as a path for communication with the outside environment, conveying and evacuating the fluids that have formed.

In particular, the percutaneous insertion of the drainage tubes is mainly and most frequently applied in situations in which an access to the thoracic cavity is not already available, or in any case in situations in which it is not easily usable.

Currently, various types of devices and methods are known for the introduction of a surgical drainage tube into the thoracic cavity of a patient; they include the trocar catheter, which is composed of a surgical drainage tube made of PVC, inside which a metal obturator is inserted which is used to give the tube the rigidity that is necessary in order to insert it through the thoracic wall.

The trocar catheter is available in various versions: with a closed tip, where the obturator remains inside the tube; with an open tip, where the obturator protrudes from the tip of the tube, maintaining an atraumatic profile; and with a sharp tip, where the obturator protrudes from the tip of the tube with a tricuspid or triangular blade profile.

The first two versions require the physician to perform a more accurate step of skin incision and creation of a passage through the intercostal space with adapted forceps (sometimes manually with the index finger, as is often described in many clinical procedure manuals).

The version with a sharp tip instead requires only the incision of the skin, after which the physician penetrates the thoracic wall by means of a calibrated push.

The presence of the inner obturator stiffens the tube in order to allow transit but at the same time increases the risk of accidental damage, particularly in the versions with a sharp tip.

Moreover, among the above-mentioned known solutions, mention can be made of small-gauge percutaneous drainages with short-medium permanence, in which access occurs through the insertion of a needle that can be of the sharp “needle-cannula” type, or of the “Verres” type provided with an atraumatic protection.

In both of these cases, the surgical drainage tube follows the pathway created by the needle, sliding inside or outside it, and entering the thoracic cavity. This type of product only allows the introduction of small-gauge drainage tubes, since there is a technical limit to the diameter of the needles that can be used.

In this group of known solutions there are also drainages that can be positioned with the Seldinger technique, i.e., by using a guide wire introduced by means of a needle. The wire, once the needle has been removed, acts as a guide for the insertion of the surgical drainage tube, typically preceded by one or more dilators in order to adapt the dimensions of the access to those of the tube.

These known solutions have some drawbacks, which include the fact that the perforation of the skin of the patient is highly traumatic.

Another drawback of these known solutions resides in the fact that they use unprotected sharp instruments. Furthermore, in almost all cases, it is necessary to proceed with a prior incision of the skin with a scalpel in order to facilitate the penetration of the surgical drainage tube. Clearly, all this increases the risks for the patient and for the physician during use.

A further drawback of these known solutions resides in the fact that it is necessary to apply intense pressure in order to achieve a penetration of the thorax of the patient. In many cases, this pressure causes damage to the patient, even up to very severe damage to internal organs, due to the inability of the physician to release the pressure in time after crossing the thoracic wall.

Another drawback of these known solutions resides in the fact that they use completely different positioning techniques depending on the type of drainage. In fact, the different configuration of the products forces the physician to learn mutually very different positioning techniques, increasing the learning curve and requiring high specialization.

A further drawback of known solutions resides in the fact that they require high manual skill and specialization of the medical staff and therefore the availability of staff qualified for these operations is a problem from an organizational point of view.

Another drawback of known solutions resides in the fact that they are limited, by their nature, to the use of drainage tubes with a tubular profile and made of rigid or semi-rigid materials and are often inadequate for positioning soft tubes, which would be more advantageous for the patient, in particular in the case of long retentions. Furthermore, known solutions are completely inadequate for drainage tubes with profiles other than the tubular one, preventing the insertion of other profiles, such as for example the splined one, which is becoming widespread in surgery.

Italian Patent Application no. IT201800001649 in the name of this same Applicant describes a percutaneous introduction device comprising a main body configured to be gripped with a single hand by the physician, like is a gun, and a retractable cutting blade which protrudes from a tubular end of said main body. The retractable cutting blade is used to create the access for the drainage tube in a safe and controlled manner. The actuation of the cutting blade is obtained by means of an internal actuation system with three springs.

This solution is capable of overcoming most of the limitations of the previously described solutions, but it is composed of a large number of components and therefore is rather complex and expensive to provide.

Furthermore, once the access has been created, it is necessary to proceed with the insertion of an adapted “introducer”, which allows the transit of the drainage tube through the layers of the skin. The introducer has the advantage of supporting any type of tube and therefore is also suitable for flexible tubes such as those made of medical silicone. Conversely, there is the drawback that in order to insert the introducer it is necessary to previously extract the device with which the access was created. This operation, besides requiring two separate accessories, exposes the muscle planes to the risk of dislocation and therefore of no longer being able to pass through the access created previously.

### SUMMARY

The aim of the present disclosure is to overcome the limitations and drawbacks of the background art described above, devising an introduction device for the percutaneous introduction of drainage tubes that is capable of creating a percutaneous access in a safe, simple and reliable manner.

Within this aim, the present disclosure provides an introduction device for the percutaneous introduction of drainage tubes the effectiveness and safety of which are highly independent from the manual skill of the operating physician.

the disclosure also provides an introduction device for the percutaneous introduction of drainage tubes that makes it possible to insert, by means of a single device, drainages having a tubular profile made of any is material, including medical silicone.

The disclosure further provides an introduction device for the percutaneous introduction of drainage tubes that makes it possible to insert, by means of a single device, drainage tubes of any gauge, comprising tubes with a gauge of more than 18 Fr (6 mm).

the disclosure also provides an introduction device for the percutaneous introduction of drainage tubes that is capable of ensuring accessibility during the entire process without the risk of losing the access created percutaneously due to the possible dislocation of the muscle and tissue planes.

Not the least advantage of the disclosure is to provide an introduction device for the percutaneous introduction of drainage tubes that is highly reliable, relatively easy to provide and has competitive costs if compared to the background art.

This aim and these and other advantages which will become more apparent hereinafter are achieved by an introduction device for the percutaneous introduction of drainage tubes, in particular thoracic drainage tubes, according to claim 1.

### BRIEF DESCRIPTION OF THE DRAWINGS

Further characteristics and advantages of the disclosure will become more apparent from the description of a preferred but not exclusive embodiment of an introduction device for the percutaneous introduction of drainage tubes, illustrated by way of non-limiting example with the aid of the accompanying drawings, wherein:

FIG. 1 is a perspective view of a possible embodiment of the introduction device according to the disclosure, in the coupled cutting configuration;

FIG. 2 is a perspective view of the introduction device of FIG. 1, in an uncoupled configuration; and

FIGS. 3 to 10 are sectional views, taken along a vertical plane, of the introduction device of FIG. 1, in various successive steps of a sequence of use.

### DETAILED DESCRIPTION OF THE DRAWINGS

With reference to the figures, the introduction device for the percutaneous introduction of drainage tubes and in particular thoracic drainage tubes, generally designated by the reference numeral 1, comprises a main body 90.

The main body 90 comprises in turn a grip portion 91 adapted to be gripped by a hand of an operator (typically the physician who performs the percutaneous introduction procedure), and a proximal portion 92 which is provided with an internal longitudinal cavity 94.

The internal longitudinal cavity 94 is preferably cylindrical and in the preferred embodiments it is closed proximally by a rear closing end plate 2 which is fixed to the proximal end of the proximal portion 92 of the main body 90.

The introduction device 1 furthermore comprises a distal tubular element 14 (which is internally hollow), which is extended longitudinally and protrudes distally from the proximal portion 92 of the main body 90.

Essentially, the introduction device 1 is configured to be gripped by an operator like a gun, with the proximal portion 92 of the main body directed toward said operator and the terminal tubular element 14 directed toward the patient. Therefore, in the present description and in the accompanying claims, the terms “proximal” and “proximally” are to be understood as, respectively, “nearest to” and “toward” the operator who grips the device 1 in the situation for use (i.e., toward the end plate 2); while the terms “distal” and “distally” mean, respectively, “furthest from” and “moving away from” the operator who grips the device 1 in the situation for use (i.e., from the end plate 2).

In the preferred and illustrated embodiment, the terminal tubular element 14 comprises a proximal part 14′, which has a larger cross-section (i.e., diameter), and a distal portion, which has a smaller cross-section and is adapted to penetrate between the tissues of the patient. The proximal part 14′ is fixed to the proximal portion 92 of the main body 90 and partially closes the internal longitudinal cavity 94 on the opposite side with respect to the rear closing end plate 2, forming essentially a distal base 71 of the internal longitudinal cavity 94.

It should be noted that the distal end of the terminal tubular element 14 tapers and has an atraumatic rounded shape, being configured to act as dilator of the access between the tissues of the patient after the cut has been performed; a slit 40 is formed in the distal end of the terminal tubular element 14 and the cutting blade 9, which will be described hereinafter, protrudes from it during use.

Going back to the general features of the introduction device 1, it comprises a longitudinally extended percussion element 11, which comprises a distal end 89 to which a cutting blade 9 is fixed. In greater detail, the cutting blade 9 is fixed in a slot formed at the distal end 89 of the percussion element 11, said distal end tapering distally in the form of a wedge.

The percussion element 11 is accommodated at least partially inside the terminal tubular element 14 and more precisely at least one distal portion of the percussion element 11 is inside the terminal tubular element 14, while a proximal part 11′ of the percussion element 11 extends also inside the internal longitudinal cavity 94 of the proximal portion 92 of the main body 90.

The percussion element 11 can slide inside the terminal tubular element 14 (and preferably also partially inside the internal longitudinal cavity 94) between a retracted position and an extracted position.

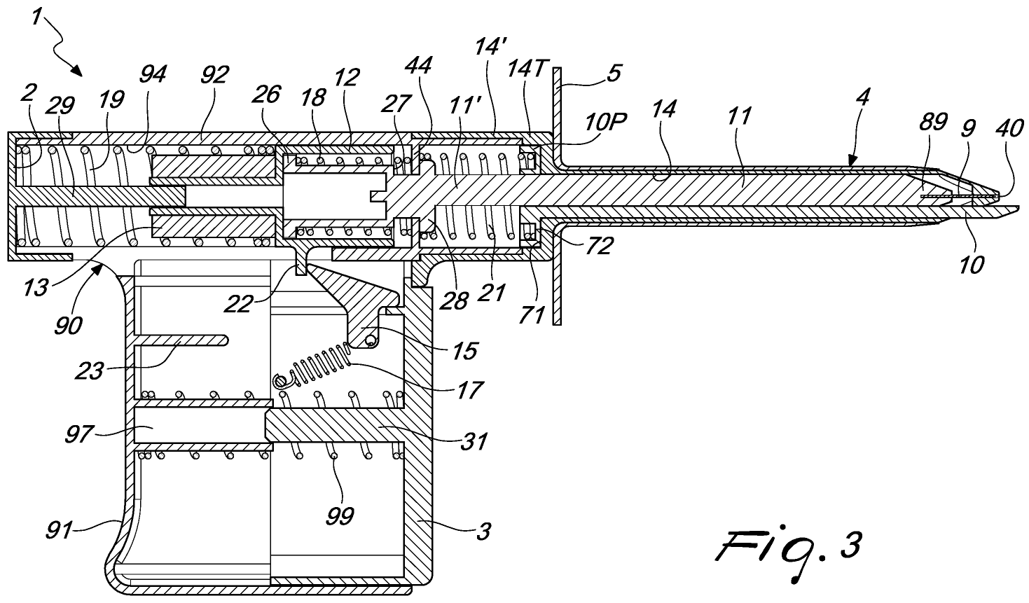

In the retracted position (shown in FIGS. 3-6) the cutting blade 9 is included (completely) inside the terminal tubular element 14, i.e., does not protrude outside it.

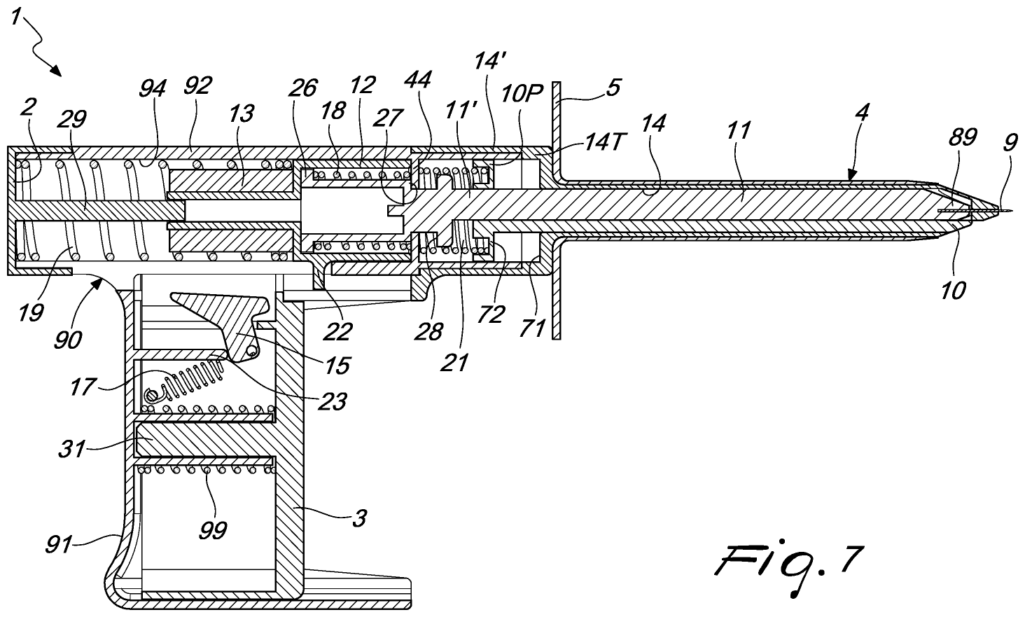

In the extracted position, the cutting blade 9 protrudes at least partially from the terminal tubular element 14, protruding from the slit 40, as shown in FIG. 7.

In practice, the cutting blade 9 is retractable and by protruding from the terminal tubular element 14 it has the function of cutting the tissues of the patient in order to create the access for the drainage tube in a safe and controlled manner.

In the preferred embodiments, the cutting blade 9 in the extracted position exits from the terminal tubular element 14 by an extent comprised between 1 mm and 4 mm, these measurements having been found by the Applicant to be optimum to ensure at the same time cutting effectiveness and safety of the patient against any accidental damage.

In greater detail, the proximal part 11′ of the percussion element 11 is provided with a first stroke limit shoulder 27 which is arranged proximally with respect to an internal abutment wall 44 and with a second stroke limit shoulder 28 which is arranged distally with respect to said internal abutment wall 44; the two stroke limit shoulders 27, 28 abut against the internal abutment wall 44, limiting the sliding of the percussion element 11: the distance between the first stroke limit shoulder 27 and the second stroke limit shoulder 28 determines the maximum stroke of the percussion element 11.

The introduction device 1 also comprises a manual actuator 3, adapted to be actuated by the hand of the operator and configured to cause the movement of the percussion element 11 from the retracted position to the extracted position, by virtue of an appropriate kinematic chain the preferred embodiment of which will be described hereinafter. Said kinematic chain passes inside the internal cavity 94, i.e., some elements that compose the kinematic chain are comprised in the internal cavity.

The manual actuator 3 is coupled to, or proximate to, the grip portion 91. In this manner the operator, by gripping the grip portion 91, can actuate is the protrusion of the cutting blade 9 by actuating the manual actuator 3 with the index and/or middle finger.

According to the disclosure, the introduction device 1 comprises a tubular introducer 4 which forms a through duct inside it.

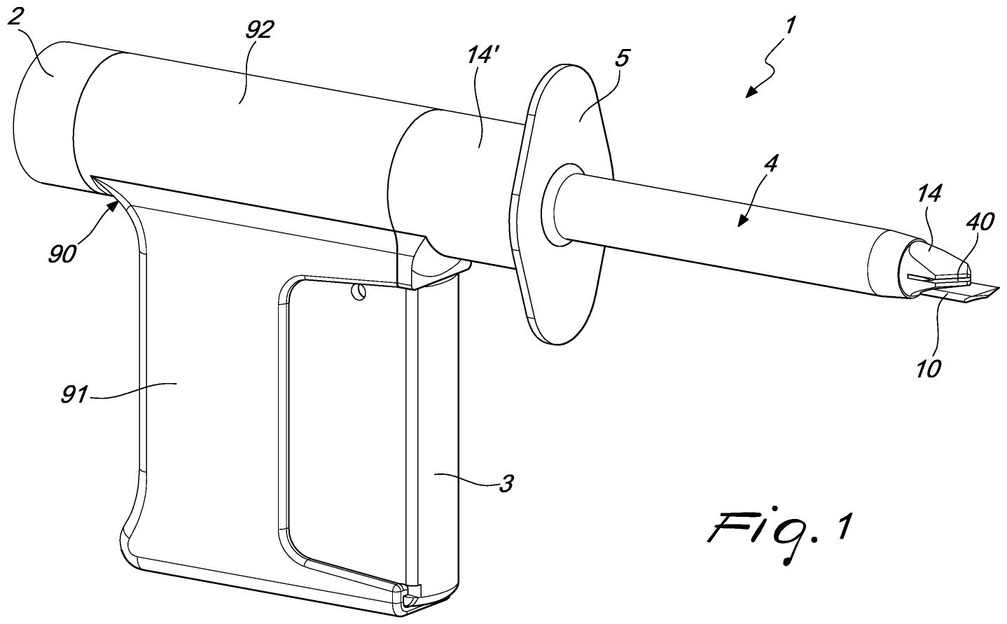

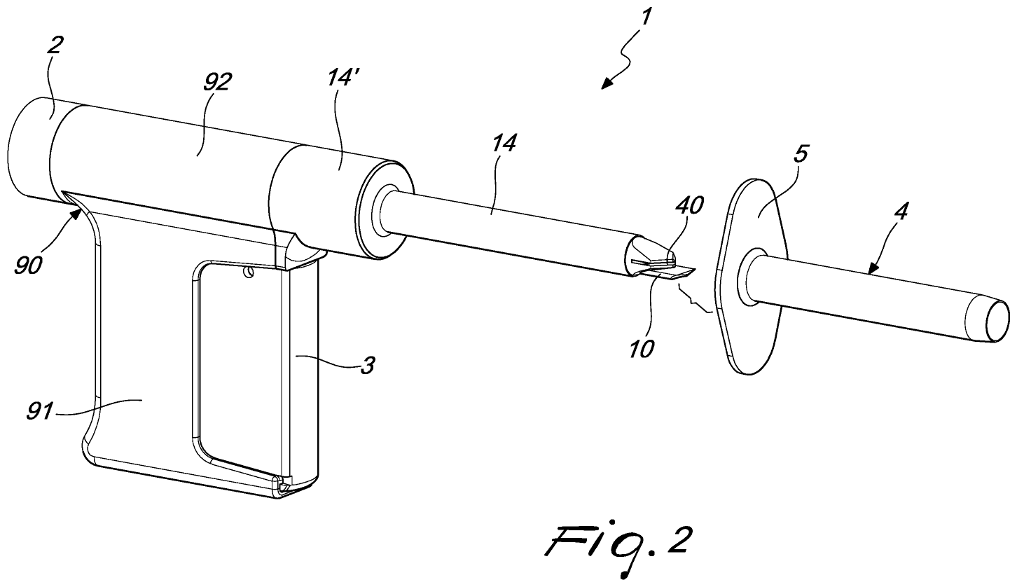

Also according to the disclosure, the introduction device 1 can be configured in a coupled cutting configuration (shown in FIG. 1) and in an uncoupled configuration (shown in FIG. 2).

In the coupled cutting configuration, the tubular introducer 4 is fitted on the terminal tubular element 14, coaxially and externally thereto, so that the distal end of the terminal tubular element 14 protrudes distally from the through duct of the tubular introducer 4 (i.e., from a distal end thereof). Therefore, the through duct of the tubular introducer 4 has a shape and dimensions which are complementary to the external ones of the terminal tubular element 14: for example, in the illustrated embodiment the tubular introducer 4 and the terminal tubular element 14 both have a circular cross-section, the external diameter of the terminal tubular element 14 being substantially equal to the inside diameter of the through duct of the tubular introducer 4.

The coupled configuration is termed herein “cutting configuration”, since in this configuration the introduction device 1 is adapted to create an access path between the tissues of a patient by means of the action of the cutting blade 9; in this manner, during said operation the terminal tubular element 14 is introduced between the tissues of the patient together with the tubular introducer 4 coupled thereto.

The tubular introducer 4 can therefore be extracted (i.e., is detachable, to removable) from the terminal tubular element 14 in order to pass from the coupled cutting configuration to the uncoupled configuration, in which the tubular introducer 4 and the terminal tubular element 14 are uncoupled, so that the through duct of the tubular introducer 4 is free for the passage of a drainage tube which can be introduced in the body of the patient through the is through duct of the tubular introducer 4.

Therefore, in the uncoupled configuration, the tubular introducer 4 is adapted to act as a guide for the percutaneous introduction of the drainage tube.

Preferably, the tubular introducer 4 comprises a stroke limiting flange 5 which is arranged at a proximal end of said tubular introducer 4 and which in the coupled cutting configuration is directed toward (or adjacent to) the proximal portion 92 of the main body 90; said stroke limiting flange 5 acts as a stroke limiter during introduction in the tissues of the patient, aids the operator in positioning and can also be used to keep the tubular introducer 4 stationary while the rest of the introduction device 1 is extracted.

Optionally, the tubular introducer 4 can be provided with a valve (preferably made of silicone) which, in the uncoupled configuration, protects the access of the through duct and, particularly in thoracic applications, prevent the exchange of air between the environment and the thoracic cavity of the patient.

According to a first optional and advantageous characteristic, the introduction device 1 also comprises a spacer 10 which has a safety function in order to prevent unintentional cutting.

The spacer 10, which comprises preferably a bar or rod or other element which is longitudinally extended, extends longitudinally within the terminal tubular element 14, in parallel to the percussion element 11.

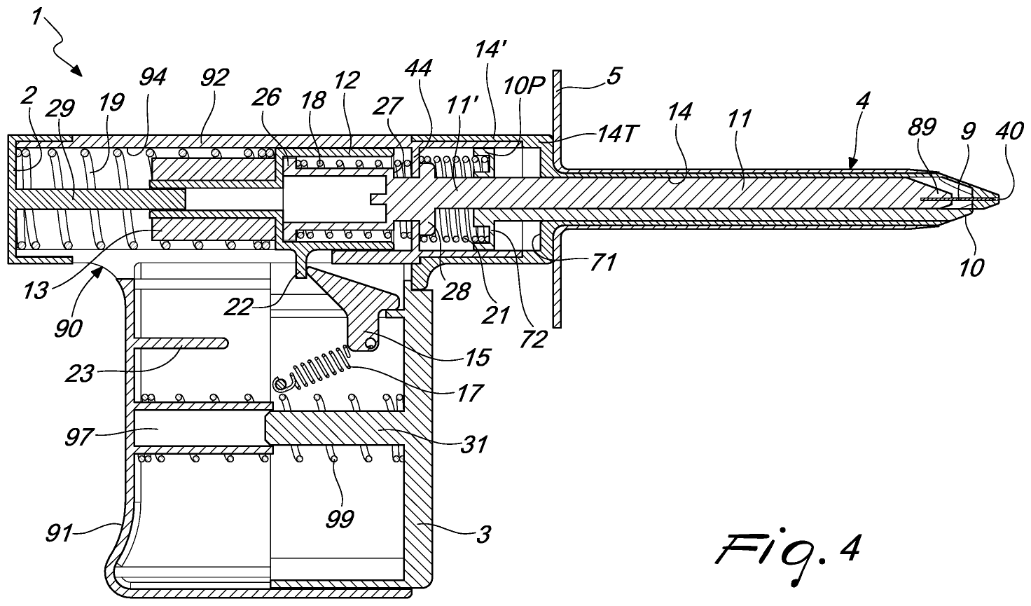

The spacer 10 can move between a safety position (shown in FIGS. 1-3), in which a distal end thereof protrudes distally out of the terminal tubular element 14, and a retracted position (shown in FIGS. 4-10), in which said distal end of the spacer 10, too, is included in the terminal tubular element 14 (i.e., it does not protrude from the terminal tubular element 14).

In the safety position, the spacer 10 protrudes from the terminal tubular element 14 by a length greater than that of the portion of the cutting blade 9 that protrudes in the extracted position.

In practice, the spacer 10, when it is in the safety position, spaces the distal end of the terminal tubular element 14 from the tissues of the patient and prevents the cutting blade 9 from cutting them.

Conveniently, the spacer 10 comprises, at its proximal end, a wider base 72 which in the safety position rests on the distal base 71 of the internal cavity 94, acting as a stroke limiter.

Preferably, the spacer 10 is pushed or pulled toward said safety position by an elastic positioning element 21 (for example a spring).

In this manner, by pushing the distal end of the terminal tubular element 14 against a surface (for example the tissues of the patient) the spacer 10 is pushed into the retracted position.

Preferably, the elastic positioning element 21 consists of a spring which is interposed between the spacer 10 and the internal abutment wall 44 which is arranged in the internal cavity 94 and pushes the spacer 10 toward the safety position.

Even more preferably, the elastic positioning element 21 is compressed between the wider base 72 of the spacer 10 and the internal abutment wall 44.

Optionally, the spacer 10 comprises a proximal portion 10P which in said safety position is arranged at an at least partially transparent portion 14T of the terminal tubular element 14 (or, as an alternative, of the proximal portion 92 of the main body 90).

Preferably, the partially transparent portion 14T corresponds to the proximal part 14′ which has a larger cross-section than the terminal tubular element 14, while the proximal portion 10P of the spacer 10 corresponds, or is adjacent, to its wider base 72.

In the preferred embodiment, the entire terminal tubular element 14 is made of substantially transparent material (preferably transparent plastic material).

At least the proximal portion 10P of the spacer 10 is opaque (for example green or of another color that is easy to identify) so as to be visible to the operator, through said at least partially transparent portion 14T, when the spacer 10 is in the safety position. In this manner the operator is informed if the cutting blade 9 is in a condition to cut tissues or not, even without seeing directly the distal end of the terminal tubular element 14.

In the preferred embodiment, the entire spacer 10 is opaque and colored.

Advantageously, the at least partially transparent portion 14T of the terminal tubular element 14 is extended over the entire circumference thereof, so that the opaque colored proximal portion 10P of the spacer 10 is visible from any angle of the introduction device 1; it should be noted that in the illustrated example said opaque proximal portion 10P is extended along a cylindrical surface.

In some embodiments, including the one shown, the introduction device 1 comprises a sliding impulse element 12, which can slide longitudinally within the internal longitudinal cavity 94 of the proximal portion 92 of the main body 90 and is configured to push the percussion element 11 toward the extracted position when the manual actuator 3 is operated.

In greater detail, in these embodiments the introduction device 1 comprises:
the sliding impulse element 12, which can slide longitudinally within the internal longitudinal cavity 94, along a longitudinal guide 29 which in the illustrated example protrudes from the rear closing end plate 2;
an elastic pusher element 19 (preferably a spring), which is to configured to push the sliding impulse element 12 toward the percussion element 11 so as to impart thereto a mechanical impulse (i.e., a push) toward the extracted position;
an elastic return element 18 (preferably a spring), which is configured to apply to the percussion element 11 a force (by pushing or is pulling it) toward the retracted position, so as to draw it back and keep it in the retracted position.

In the illustrated embodiment, the elastic return element 18 is compressed between the internal abutment wall 44 and a proximal shoulder 26 of the percussion element 11, said proximal shoulder 26 being positioned at a proximal end of the percussion element 11, proximate with respect to the internal abutment wall 44.

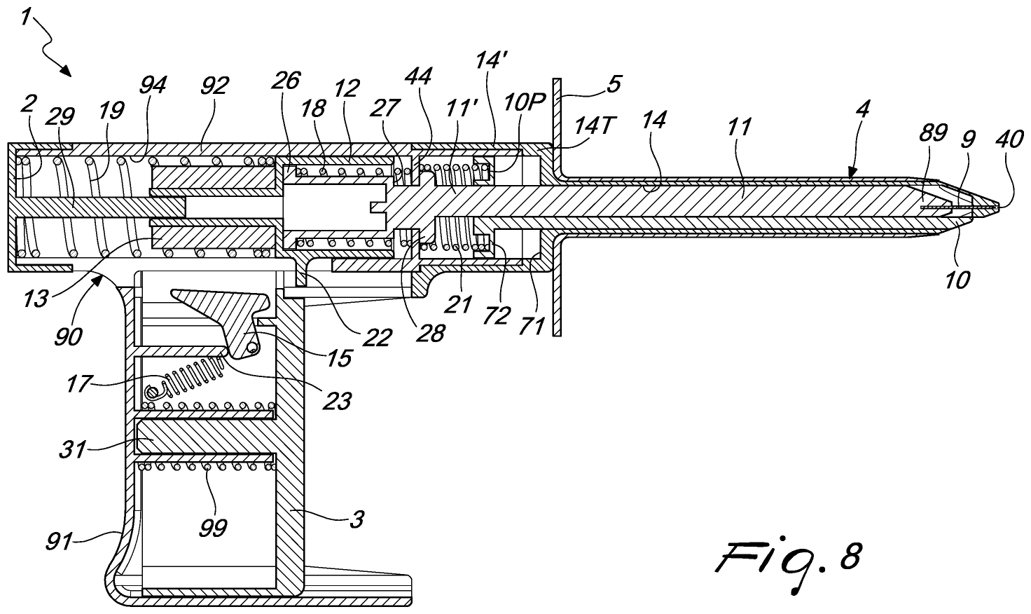

As already mentioned, there is also the manual actuator 3, which is adapted to be actuated by the hand of the operator and can be moved between a stroke start position (FIG. 3) and a stroke end position (FIG. 8).

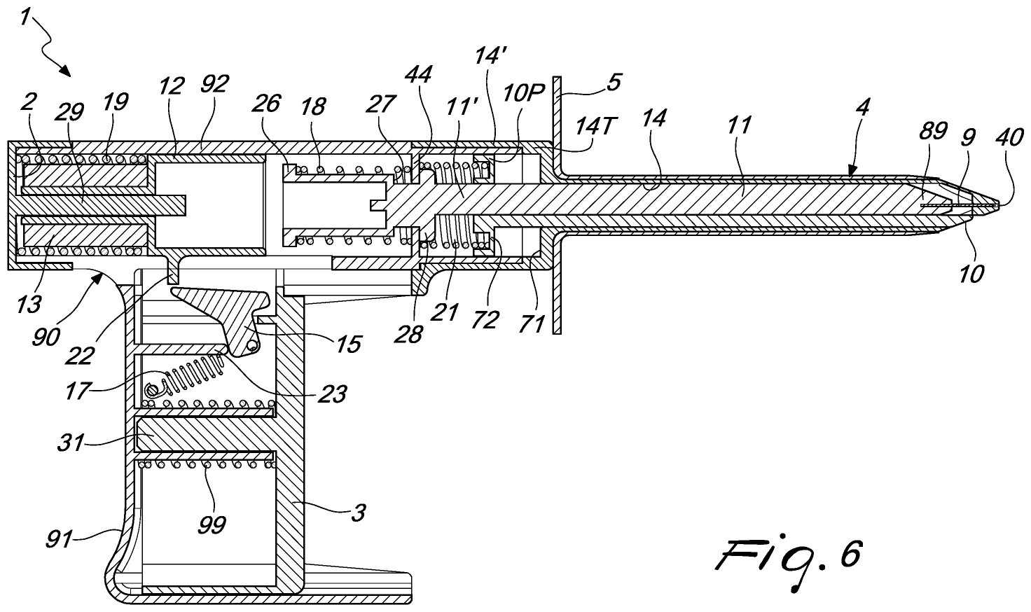

The manual actuator 3 is configured to induce a movement of the sliding impulse element 12 (for example by dragging it) toward a position for loading the elastic pusher element 19; in the preferred embodiment, in which the elastic pusher element 19 consists of a spring which pushes the sliding impulse element 12, said loading position corresponds to a compression position of the elastic pusher element 19 as shown in FIG. 6.

According to another optional and advantageous characteristic, the sliding impulse element 12 is coupled to an inertial loading element 13 which has a predetermined weight and is adapted to increase the mechanical impulse provided by the sliding impulse element 12 to the percussion element 11.

In order to ensure an optimum cutting effectiveness of the cutting blade 9, the inertial loading element 13 has a weight preferably comprised between 15 g and 60 g.

In the preferred embodiment, the sliding impulse element 12 comprises a cylindrical or cup-like body the open cavity of which is directed toward the percussion element 11 so that the base of said open cavity rests on a proximal base of the percussion element 11 in order to push it.

The sliding impulse element 12 further comprises a portion which is proximally elongated and has a smaller diameter than the cylindrical or cup-like body, on which the inertial loading element 13 is keyed or in any case fixed.

It should be noted that the proximally elongated portion is provided with a tunnel which is slidingly coupled to the longitudinal guide 29 so that it can slide thereon.

It should be noted, moreover, that the elastic pusher element 19 extends from the rear closing end plate 2 to the cylindrical or cup-like body of the sliding impulse element 12, passing externally to the inertial loading element 13.

As regards the manual actuator 3, it preferably comprises, or is constituted by, a slider positioned on the distal face of the grip portion 91 so as to be actuatable by the index and/or middle finger of the operator.

Conveniently, said slider comprises at least one sliding stem 31 which can slide in a guiding channel 97.

Preferably, the introduction device 1 comprises an engagement member 15 which is configured to retain translationally (i.e., render integral) the sliding impulse element 12 and the manual actuator 3 (i.e., the slider) during the movement thereof from the stroke start position to the stroke end position and to release the sliding impulse element 12 when the manual actuator 3 reaches the stroke end position.

In greater detail, with reference to the particular preferred and illustrated embodiment, the engagement element 15 is coupled to the manual actuator 3 so that it translates integrally with the manual actuator 3 and can be rotated between an engagement position (shown in FIGS. 3-5), in which it rests on an abutment rib 22 or flange of the sliding impulse element 12, and a disengagement position (shown in FIG. 6), in which it releases said abutment rib 22. The engagement member 15 is moreover coupled to an elastic engagement element 17 which forces said engagement member 15 into the engagement position (for example by pulling it).

Such elastic engagement element 17 is preferably a spring which is fixed with a first end to an arm of the engagement member 15 and with a second end to the slider of the manual actuator 3.

It should be noted that in the illustrated example the engagement member 15 has an asymmetric shape and has a first arm, which contacts the abutment rib 22 of the sliding impulse element 12, and a second arm, which is angled with respect to the first one and to which the elastic engagement element 17 is fixed.

There is furthermore, inside the grip portion 91, a stroke limit element 23 (such as for example a protruding wall or a protrusion) which induces the rotation of the engagement element 15 from the engagement position to the disengagement position when the manual actuator 3 reaches the stroke end position. In practice, the stroke limit element 23 contacts the second arm of the engagement member 15, causing its rotation.

Inside the grip portion 91 there is furthermore a return spring 99, which is configured to push the manual actuator 3 from the stroke end position to the stroke start position and is compressed when the manual actuator 3 is in the stroke end position. This return spring 99 is preferably held in position by an internal shaped portion or supporting element comprised in the grip portion 91. In the illustrated embodiment, said supporting element is the same element that forms the guiding channel 97 on its inside; in this case, the return spring 99 is coaxial with the guiding channel 97 and the sliding stem 31.

The operation of the introduction device 1 is clear and evident from what has been described. FIGS. 3 to 10 show in detail a sequence of operation of the preferred embodiment.

FIG. 3 shows the starting or “safety” position of the device 1. The manual actuator 3 is in the stroke start position, the percussion element 11 is held in retracted position by the elastic return element 18 so that the cutting blade 9 is completely hidden inside the terminal tubular element 14. The spacer 10 is in the safety position and therefore its opaque colored proximal portion 10P is visible to the operator through the transparent portion 14T. Finally, the engagement member 15 is in the engagement position.

This is the position in which the device 1 is given to the operator, ready to be used and therefore advantageously available in emergency and urgent cases where it would be complicated to perform preliminary assemblies.

The operator then rests the distal end of the terminal tubular element 14 on the skin of the patient at the point previously selected as suitable for entry (for example, by means of an ultrasound and/or X-ray survey). By resting the distal end one obtains the backward movement of the spacer 10 into the retracted position due to the resistance offered by the tissues (skin and muscle bundles) of the patient, as shown in FIG. 4.

Keeping the tip of the terminal tubular element 14 of the device 1 firmly pressed in position, the operator pushes or pulls the manual actuator 3 toward the stroke end position by using the index or middle finger or both, depending on which option is more comfortable. The grip portion 91 and the actuation of the manual actuator 3 are suitable for both right-handed and left-handed operators. The movement of the manual actuator 3 compresses the return spring 99. The movement of the actuator 3, toward the stroke end position, entails the parallel backward movement of the sliding impulse element 12, by virtue of the action of the engagement member 15 that pushes against the abutment rib or flange 22.

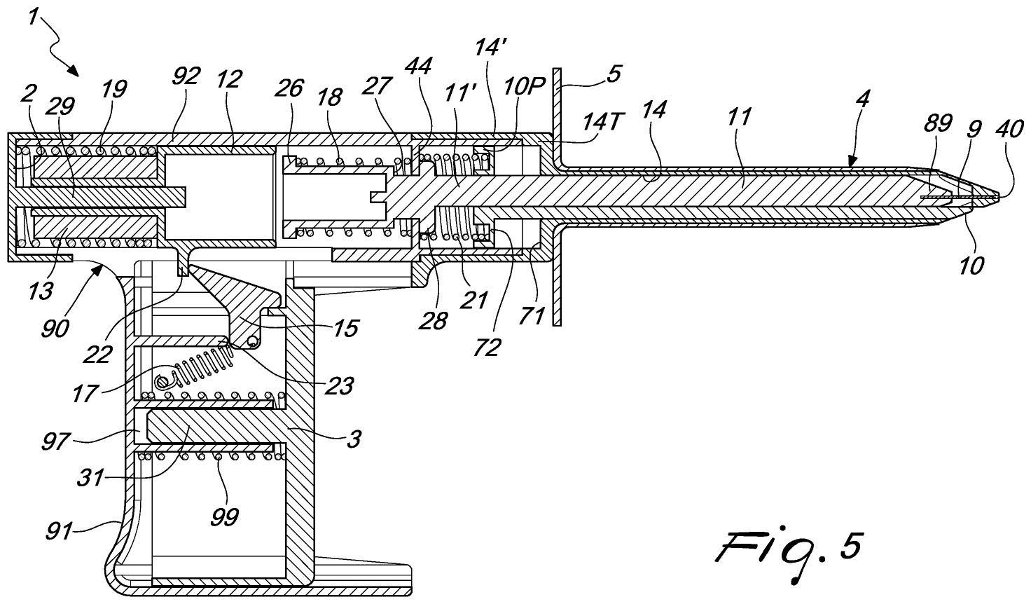

The manual actuator 3 then approaches the stroke end position, as shown in FIG. 5, and the engagement member 15 meets, in its second arm (i.e., in the lower portion), the stroke limit element 23, which exerts a pressure exactly on the lower part of said engagement member 15, starting its counterclockwise rotation. Such rotation has the effect of releasing the engagement member 15 from the abutment flange 22.

At the same time, in the proximal portion 92 of the main body 90 of the device, the sliding impulse element 12 also approaches the stroke end, causing the compression of the elastic pusher element 19.

FIG. 6 shows the manual actuator 3 which has reached the stroke end and the engagement member 15 rotated until the abutment rib or flange 22 of the sliding impulse element 12 is freed completely. The sliding impulse element 12 is therefore free for release, toward the distal part of the device 1, pushed by the elastic pusher element 19.

FIG. 7 shows the step of release of the sliding impulse element 12, which is free to slide toward the distal part, pushed by the elastic pusher element 19, which releases all the accumulated elastic potential energy. The sliding impulse element 12 thus makes contact with the proximal base of the percussion element 11. The inertial loading element 13 gives the sliding impulse element 12 an inertia that is sufficient to compress the elastic return element 18 and therefore to make the percussion element 11 slide to the extracted position, in which the stroke limit shoulder 27 abuts against the internal abutment wall 44.

Such forward stroke allows the cutting blade 9 to protrude from the distal end of the terminal tubular element 14, from the slit 40.

The cutting blade 9 can therefore perform the incision of the tissues that are along its path with a clear and well-defined cut, due to the inertia and to the limited protrusion of said blade 9.

Subsequently, the elastic return element 18 returns the percussion element 11 to the retracted position, consequently causing the cutting blade 9 to retract into the retracted safety position, as shown in FIG. 8.

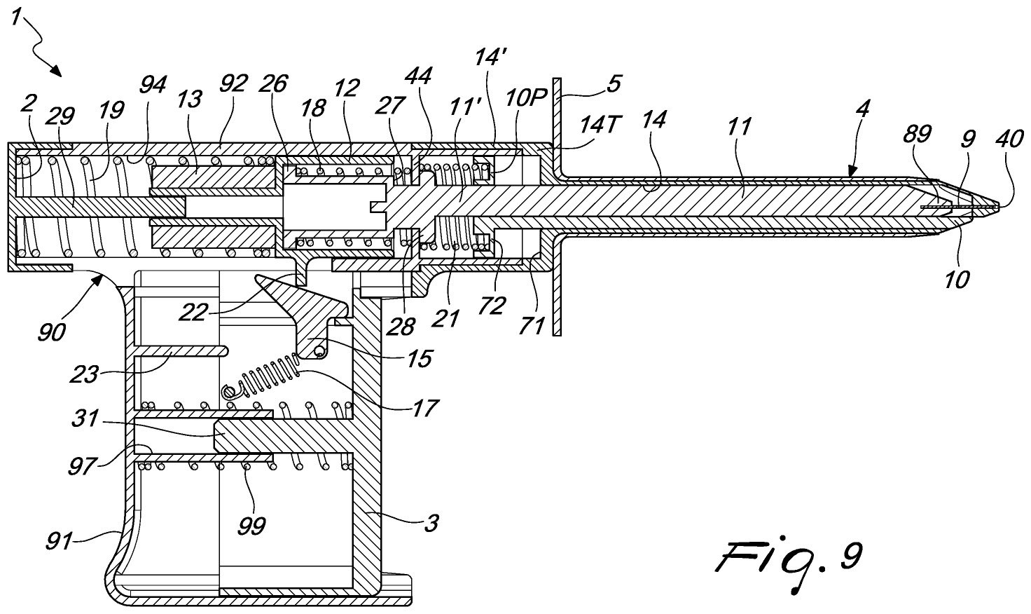

At this point, as shown in FIG. 9, the operator releases the manual actuator 3, which is pushed toward the stroke start position by the return spring 99. During the return stroke, the engagement member 15 rotates in its starting position due to the traction of the elastic engagement element 17. The engagement member 15 retracts integrally with the manual actuator 3 toward the stroke start position.

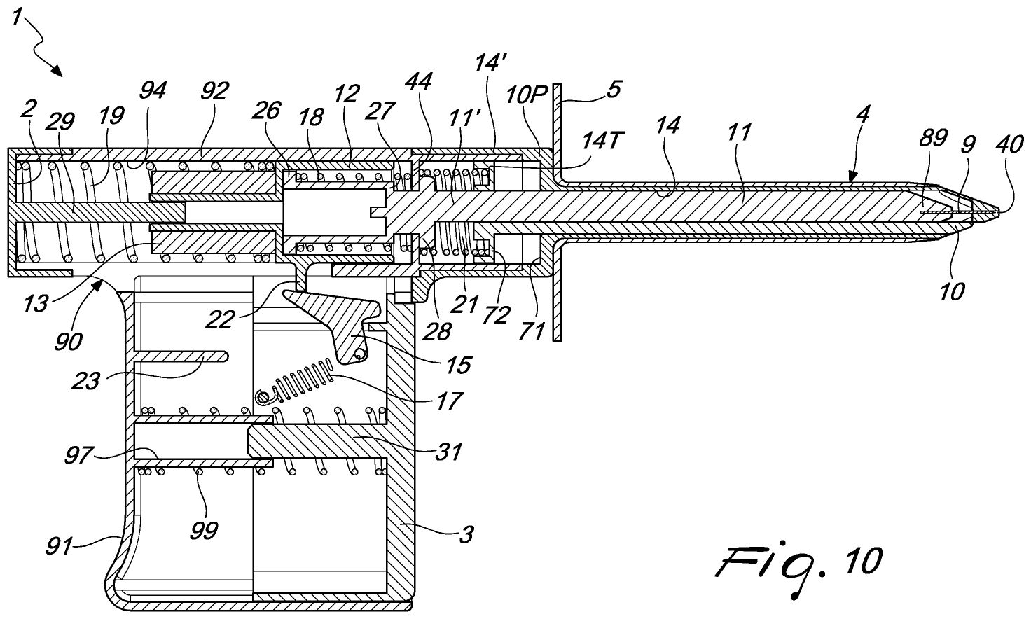

As shown in the subsequent FIG. 10, during the movement of the actuator 3 toward the stroke start position the engagement member 15 encounters the abutment rib or flange 22 of the sliding impulse element 12, which causes a counterclockwise rotation of said engagement member 15. This rotation has the effect of allowing the engagement member 15 to be able to move beyond the abutment flange 22 and return to the starting engagement position, ready for a subsequent cutting procedure.

The cutting procedure can be performed multiple times, repeating the sequence described above, until the operator deems that he has passed the thoracic or abdominal wall. At that point, the resistance of the underlying tissues is no longer such as to contrast the thrust of the elastic positioning element 21 and therefore the spacer 10 can extend in a safety position, exceeding the maximum extraction length of the cutting blade 9. In this manner, any subsequent actuation of the blade 9 by the operator will not cause any damage to the surrounding tissues and/or organs, due to protection by the spacer 10. The operator sees this safety condition through the adapted viewing element described previously.

Once the distal end of the terminal tubular element 14 has penetrated beyond the external tissues and the muscle bundles, the operator can end the insertion of the entire distal length of the tubular element 14 of the device 1 at most up to the stroke limiting flange 5, which determines the maximum insertable length.

Such stroke limiting flange 5 is used by the operator to keep the tubular introducer 4 in place while the remaining part of the device 1 is extracted by applying a slight traction to the main body 90.

In this manner, the tubular introducer 4 remains as a connection means between the outside and the inside of the body of the patient.

Catheters with a tubular profile of various types and made of different materials can be introduced easily through the internal through duct of the tubular introducer 4. The structure of the access created is such as to be particularly advantageous for drainage tubes made of flexible material, such as medical-grade silicone, and for splined round profiles; in both of these situations, the low rigidity of the material and/or the impossibility to insert an internal obturator, would render positioning with non-surgical techniques impossible.

At the end of the positioning of the drainage tube, the tubular introducer 4 can be easily extracted by following the longitudinal axis of said drainage up to its free end.

In practice it has been found that the introduction device for the percutaneous introduction of drainage tubes, according to the present disclosure, achieves the intended aim and objects, since it makes it possible to create a percutaneous access in a safe, simple and reliable manner.

Another advantage of the introduction device, according to the disclosure, resides in the fact that its effectiveness and safety are highly independent of the manual skill of the physician operator.

An additional advantage of the introduction device, according to the disclosure, resides in the fact that it allows the insertion, by means of a single device, of drainages with a tubular profile made of any material, including medical silicone.

Another advantage of the introduction device, according to the disclosure, resides in the fact that it allows the insertion, by means of a single device, of drainage tubes of any gauge, comprising tubes with a gauge higher than 18 Fr (6 mm).

Another advantage of the introduction device, according to the disclosure, resides in the fact that it is capable of ensuring accessibility during the entire process without the risk of losing the access created percutaneously due to the possible dislocation of the muscle and tissue planes.

Another advantage of the introduction device, according to the disclosure, resides in the fact that it is highly reliable, relatively easy to provide and has competitive costs if compared with the background art.

The introduction device for the percutaneous introduction of drainage tubes thus conceived is susceptible of numerous modifications and variations, all of which are within the scope of the accompanying claims.

All the details may furthermore be replaced with other technically equivalent elements.

In practice, the materials used, as well as the contingent shapes and dimensions, may be any according to the requirements and the state of the art.

1-12. (canceled)

13. An introduction device for percutaneous introduction of thoracic drainage tubes, the introduction device comprising:
a main body, which comprises a grip portion adapted to be gripped by a hand of an operator, and a proximal portion provided with an internal longitudinal cavity;
a terminal tubular element, which is extended longitudinally and distally from said proximal portion of the main body;
a longitudinally extended percussion element, which comprises a distal end to which a cutting blade is fixed and which can slide within said terminal tubular element between a retracted position, in which said cutting blade is included within said terminal tubular element, and an extracted position, in which the cutting blade protrudes at least partially from said terminal tubular element;
and further comprising a tubular introducer which forms inside it a through duct,
and wherein said introduction device can be configured in a coupled cutting configuration, in which the tubular introducer is fitted on said terminal tubular element, and in an uncoupled configuration, in which the tubular introducer and the terminal tubular element are uncoupled, so that the through duct of the tubular introducer is free for a passage of a drainage tube;
in said coupled cutting configuration the introduction device being adapted to create an access path between tissues of a patient by means of said cutting blade;
said tubular introducer being extractable from the terminal tubular element in order to pass from said coupled cutting configuration to said uncoupled configuration;
in said uncoupled configuration the tubular introducer being adapted to act as a guide for the percutaneous introduction of the drainage tube.

14. The introduction device according to claim 13, wherein the tubular introducer comprises a stroke limiting flange which is arranged at a proximal end of the tubular introducer and wherein in said coupled cutting configuration the tubular introducer is directed toward the proximal portion of the main body.

15. The introduction device according to claim 14, further comprising:
a sliding impulse element, which can slide longitudinally within said internal longitudinal cavity,
an elastic pusher element, which is configured to push said sliding impulse element toward the percussion element so as to impart thereto a mechanical impulse toward the extracted position,
an elastic return element, which is configured to apply to the percussion element a force toward the retracted position so as to draw it back and keep it in said retracted position,
a manual actuator, adapted to be actuated by the hand of the operator;
said manual actuator being configured to induce a movement of the sliding impulse element toward a position for loading the elastic pusher element.

16. The introduction device according to claim 15, wherein the sliding impulse element is coupled to an inertial loading element which has a predetermined weight and is adapted to increase the mechanical impulse provided by the sliding impulse element to the percussion element.

17. The introduction device according to claim 16, wherein the inertial loading element has a weight comprised between 15 g and 60 g.

18. The introduction device according to claim 15, wherein the manual actuator is configured to be moved between a stroke start position and a stroke end position,
wherein the introduction device comprises an engagement element which is configured to couple in translation the sliding impulse element and the manual actuator during the movement thereof from the stroke start position to the stroke end position and to release said sliding impulse element when the manual actuator reaches the stroke end position.

19. The introduction device according to claim 18, wherein the engagement element s coupled to the manual actuator so that said engagement element translates integrally with the manual actuator and can be rotated between an engagement position, in which the engagement element rests on an abutment rib of the sliding impulse element, and a disengagement position, in which the engagement element releases said abutment rib;
said engagement element being coupled to an elastic engagement element which forces said engagement member into said engagement position; the introduction device comprising a stroke limit element which induces the rotation of the engagement element from the engagement position into the disengagement position when the manual actuator reaches the stroke end position.

20. The introduction device according to claim 13, further comprising a spacer which is extended longitudinally within the terminal tubular element in parallel to the percussion element and can move between a safety position, in which a distal end thereof protrudes distally out of said terminal tubular element, and a retracted position, in which said distal end of the spacer is included in the terminal tubular element.

21. The introduction device according to claim 20, further comprising an elastic positioning element which pushes or pulls the spacer toward said safety position.

22. The introduction device according to claim 21, wherein said elastic positioning element is a spring which is interposed between the spacer and an internal abutment wall which is arranged in a proximal portion of the main body and pushes the spacer toward said safety position.

23. The introduction device according to claim 22, wherein the spacer comprises a proximal portion which in said safety position is arranged at an at least partially transparent portion of the terminal tubular element or of the proximal portion of the main body; said proximal portion of the spacer element being opaque so that it is visible to the operator, through said at least partially transparent portion, when said spacer is in said safety position.

24. The introduction device according to claim 22, wherein the terminal tubular element comprises:
a proximal part, which has a larger cross-section and is fixed to the proximal portion of the main body and partially closes the internal longitudinal cavity, and
a distal portion, which has a smaller cross-section and is adapted to penetrate between the tissues of a patient and on which the tubular introducer is fitted in the coupled cutting configuration.
